(19) United States
(12) Patent Application Publication
(10) Pub. No.: US 2021/0077026 A1
(43) Pub. Date: Mar. 18, 2021

(54) NON-INVASIVE GLUCOSE MONITORING SYSTEM

(71) Applicant: Medika Healthcare Co., Ltd., Fremont, CA (US)

(72) Inventors: Roy SingFatt Chin, Fremont, CA (US); Robin Bek, Campbell, CA (US)

(21) Appl. No.: 17/071,898

(22) Filed: Oct. 15, 2020

Related U.S. Application Data
(62) Division of application No. 15/691,694, filed on Aug. 30, 2017, now abandoned.
(60) Provisional application No. 62/382,088, filed on Aug. 31, 2016.
(60) Provisional application No. 62/398,466, filed on Sep. 22, 2016.

(51) Int. Cl. A61B 5/00; A61B 5/145; A61B 5/1455

(52) U.S. Cl. CPC A61B 5/6826; A61B 5/14532; A61B 5/01; A61B 5/725; A61B 5/1455

(57) ABSTRACT

A blood monitoring system employs a finger holder capable of receiving and stabilizing fingers of various sizes. A blood monitoring system employs a bandpass filter array capable of reducing optical crosstalk.

### TECHNICAL FIELD

Embodiments of this disclosure relate generally to systems and methods for measuring constituents in a sample. In particular, various embodiments of a glucose monitoring system allowing noninvasive measurement of blood glucose are described.

### BACKGROUND

Diabetes is a disease in which blood glucose levels are above normal. Most of the food people eat is turned into glucose or sugar for energy. The pancreas, an organ that lies near the stomach, makes insulin to help glucose enter body's cells. When people have diabetes, either the pancreas does not make enough insulin or the body cannot use their own insulin as well as it should. This causes sugar to build up in the blood.

Diabetes can cause serious health complications including heart disease, blindness, kidney failure, and lower-extremity amputations. The most serious problem caused by diabetes is heart disease. People with diabetes are more than twice likely to have heart disease or stroke. However, people with diabetes may not have the usual signs or symptoms of a heart attack. Therefore, the best way is to work with a healthcare team to keep the blood glucose, blood pressure, and cholesterol levels in the normal range. People with diabetes should work with healthcare providers which can monitors their diabetes control or help them learn to manage diabetes.

Conventionally, healthcare providers and patients use needle-prick devices to take blood from patients' fingers for testing blood glucose. The painful nature of drawing blood through skins discourages people from frequent testing of blood glucose, causing patients who have diabetes to not be as diligent as they should be for good blood glucose control. Accordingly, there exists a continuing need for glucose monitoring systems and methods that allow noninvasive measurement of blood glucose.

### SUMMARY

Provided herein is a finger holder which can be used in a blood monitoring system. The finger holder comprises a first holder portion having a first end, a second end, and an inner surface extending from the first end to the second end, a second holder portion having a first end, a second end, and an inner surface extending from the first end to the second end, and torsion spring. The first ends of the first and second holder portions form an opening to admit a finger to be received between the inner surfaces of the first and second holder portions. The torsion spring comprises a first arm coupled to the first holder portion providing a force to urge the first holder portion to the second holder portion, a second arm anchored to a support member, and a spring coil retained by a retaining post secured to the support member allowing the first holder portion to rotate about the retaining post thereby increasing or decreasing a size of the opening admitting the finger.

In some embodiments, the first holder portion may further comprise a retaining structure at the second end. The retaining structure is provided with a through slot receiving the retaining post, allowing the first holder portion to translate relative to the second holder portion and the retaining post, thereby increasing or decreasing a space between the inner surfaces of the first and second holder portions.

In some embodiments, the spring coil may comprise a first coil section and a second coil section spaced apart and connected by the second arm. The first and second coil sections are disposed outside of the through slot of the retaining structure and retained by the retaining post.

In some embodiments, the first holder portion may further comprise an enclosure for enclosing a light source.

In some embodiments, the first holder may further comprise a stopper at the second end preventing the finger from extending beyond the finger holder.

In some embodiments, the second holder portion may further comprise a temperature sensor for detecting the temperature of the finger.

In some embodiments, the first holder portion may be provided with an aperture allowing light passing through to illuminate the finger, and the second holder portion may be provided with an aperture allowing light attenuated by the finger to exit through.

In some embodiments, the second holder portion may further comprise a ridge on the inner surface along the aperture of the second holder portion to position or stabilize a fingertip at the aperture of the second holder portion.

In some embodiments, the first and second holder portions may each comprise a finger pad constructed from a material comprising polyurethane or liquid silicone rubber.

Also provided herein is a bandpass filter array which can be used in a blood monitoring system. The bandpass filter array comprises a plurality of bandpass filters arranged side by side in an array and a plurality of light blocking layers in between neighboring bandpass filters. Each of the plurality of bandpass filters is configured to transmit light of a band of wavelengths, comprises a first end facing incident light, a second end exiting transmitted light, and a first side and a second side extending from the first end to the second end. The first and second sides of at least one of the plurality of bandpass filters are chamfered at the second end of the at least one of the plurality of bandpass filters. Each of the plurality of light blocking layers extends from the first end to the second end of the plurality of bandpass filters.

In some embodiments, each of the plurality of bandpass filters may be chamfered at the second end of each of the plurality of bandpass filters.

In some embodiments, the first and second sides of the at least one of the plurality of bandpass filters are chamfered with an angle ranging from 10 to 80 degrees, or are chamfered with an angle ranging from 30 to 60 degrees, or are chamfered with an angle of about 45 degrees.

In some embodiments, the first and second sides of each of the plurality of bandpass filters are chamfered with an angle ranging from 10 to 80 degrees, or are chamfered with an angle ranging from 30 to 60 degrees, or are chamfered with an angle of about 45 degrees.

In some embodiments, each of the plurality of bandpass filters has a transmission center wavelength different from a transmission center wavelength of a neighboring bandpass filter.

In some embodiments, the transmission center wavelengths of the plurality of bandpass filters are spread across a wavelength range from 700 to 1040 nanometers.

In some embodiments, the transmission center wavelengths of the plurality of bandpass filters are spread across the wavelength range in a successively increased or decreased order.

In some embodiments, the bandpass filter array may comprise 35 bandpass filters. Each of the 35 bandpass filters has a different transmission center wavelength. The transmission center wavelengths of the 35 bandpass filters may be spread from 700 to 1040 nanometers with a wavelength step of up to 10 nanometers.

In some embodiments, the bandpass filter array may further comprise a holder including a plurality of walls defining an array of cavities. The plurality of bandpass filters may be disposed in the array of cavities. In some embodiments, the holder may be constructed from a light blocking material.

Also provided herein is an optical apparatus comprising a collimation lens collimating incoming light, a bandpass filter array selectively transmitting the collimated light, and a detector array optically coupled to the bandpass filter array detecting light selectively transmitted by the bandpass filter array and generating output signals indicative of intensities of the light detected. The bandpass filter array comprises a plurality of bandpass filters and a plurality of light blocking layers in between neighboring bandpass filters, the detector array comprises a plurality of light-detection elements each corresponding to one of the plurality of bandpass filters. Each of the plurality of bandpass filters has a first end distal to the detector array, a second end proximal to the detector array, and a first side and a second side extending from the first end to the second end. The first and second sides of at least one of the plurality of bandpass filters are chamfered at the second end, thereby leading the light selectively transmitted by the at least one of the plurality of bandpass filters to the corresponding light-detection element.

Further provided is a blood monitoring system comprising a finger holder disclosed herein. The blood monitoring system comprises a light source producing light beams, a finger holder configured to hold a finger to be irradiated by the light beams, a detector array detecting light attenuated by the finger and generating output signals indicative of intensity of the light detected, and a processor determining a characteristic of a blood constituent in the finger based on the generated output signals. The finger holder comprises a first holder portion having a first end, a second end, and an inner surface extending from the first end to the second end, a second holder portion having a first end, a second end, and an inner surface extending from the first end to the second end, and a torsion spring. The first ends of the first and second holder portions form an opening to admit the finger to be received between the inner surfaces of the first and second holder portions. The torsion spring comprises a first arm coupled to the first holder portion providing a force to urge the first holder portion to the second holder portion, a second arm anchored to a support member, and a spring coil retained by a retaining post secured to the support member allowing the first holder portion to rotate about the retaining post thereby increasing or decreasing a size of the opening admitting the finger.

In some embodiments, the blood monitoring system may further comprise a casing enclosing the light source, the finger holder, the detector array, and the processor inside.

In some embodiments, the light source may be an incandescent light source.

In some embodiments, the processor may comprise a duo core processor.

Further provided is a blood monitoring system comprising a bandpass filter array disclosed herein. The blood monitoring system comprises a light source producing light beams having a range of wavelengths, a finger holder configured to hold a finger in operation, a collimation lens collimating light transmitted through the finger, a bandpass filter array selectively transmitting the collimated light, a detector array optically coupled to the bandpass filter array detecting light selectively transmitted by the bandpass filter array and generating output signals indicative of intensities of the light detected, and a processor determining a characteristic of a blood constituent in the finger based on the generated output signals. The bandpass filter array comprises a plurality of bandpass filters and a plurality of light blocking layers in between neighboring bandpass filters. The detector array comprises a plurality of light detection elements each corresponding to one of the plurality of bandpass filters. Each of the plurality of bandpass filters has a first end distal to the detector array, a second end proximal to the detector array, and a first side and a second side extending from the first end to the second end. The first and second sides of at least one of the plurality of bandpass filters are chamfered at the second end, thereby leading the light selectively transmitted by the at least one of the plurality of bandpass filters to the corresponding light-detection element.

In some embodiments, the light source may be an incandescent light source.

In some embodiments, the processor may comprise a duo core processor.

In some embodiments, the blood monitoring system may comprise a casing enclosing the light source, the finger holder, the collimation lens, the bandpass filter array, the detector array, and the processor inside.

This Summary is provided to introduce selected embodiments in a simplified form and is not intended to identify key features or essential characteristics of the claimed subject matter, nor is it intended to be used as an aid in determining the scope of the claimed subject matter. The selected embodiments are presented merely to provide the reader with a brief summary of certain forms the invention might take and are not intended to limit the scope of the invention. Other aspects and embodiments of the disclosure are described in the section of Detailed Description.

### BRIEF DESCRIPTION OF THE DRAWINGS

These and various other features and advantages will become better understood upon reading of the following detailed description in conjunction with the accompanying drawings and the appended claims provided below, where:

FIG. 1 is a simplified block diagram illustrating an exemplary glucose monitoring system according to embodiments of the disclosure.

FIG. 2 is a cutaway view of an exemplary glucose monitoring system showing components according to embodiments of the disclosure.

FIG. 3 schematically shows the use of an exemplary glucose monitoring system according to embodiments of the disclosure.

FIG. 4 schematically shows an exemplary finger holder and an exemplary optical bench included in a glucose monitoring system according to embodiments of the disclosure.

FIG. 5 is a cutaway view of an exemplary finger holder and an exemplary optical bench included in a glucose monitoring system.

FIG. 6 is a top perspective view of an upper holder portion according to embodiments of the disclosure.

FIG. 7 is a bottom perspective view of an upper holder portion according to embodiments of the disclosure.

FIG. 8 is a top perspective view of a lower holder portion according to embodiments of the disclosure.

FIG. 9 is a bottom perspective view of a lower holder portion according to embodiments of the disclosure.

FIG. 10 is an exploded view of an upper holder portion and a torsion spring according to embodiments of the disclosure.

FIGS. 11 and 12 schematically show assembling of a torsion spring with an upper holder portion according to embodiments of the disclosure.

FIG. 13 schematically shows assembling of an upper holder portion-torsion spring in a glucose monitoring system according to embodiments of the disclosure.

FIG. 14 schematically shows a finger stabilized in a finger holder according to embodiments of the disclosure.

FIG. 15 is an exploded view of an exemplary optical bench according to embodiments of the disclosure.

FIG. 16 is a perspective view of an exemplary bandpass filter array coupled with a detector array according to embodiments of the disclosure.

FIG. 17 is a side cross-sectional view of the bandpass filter array coupled with a detector array shown in FIG. 16.

FIG. 18 is an enlarged partial view of the bandpass filter array coupled with a detector array shown in FIG. 17.

FIG. 19 is an enlarged partial view of a bandpass filter, light blocking layers, and a light-detection element according to embodiments of the disclosure.

FIG. 20 is a block diagram showing an exemplary bandpass filter array coupled to a detector array according to embodiments of the disclosure.

FIG. 21 is a perspective view of an exemplary bandpass filter holder according to embodiments of the disclosure.

### DETAILED DESCRIPTION

Various embodiments of a blood monitoring system are described. It is to be understood that the disclosure is not limited to the particular embodiments described. An aspect described in conjunction with a particular embodiment is not necessarily limited to that embodiment and can be practiced in any other embodiments. For example, while various embodiments of the disclosure are described in connection with a system for monitoring blood glucose, it will be appreciated that the disclosed embodiments can be used for measuring other constituents in a sample fluid such as blood oxygen, cholesterol, and hemoglobin levels etc.

Various embodiments are described with reference to the figures. It should be noted that some figures are not necessarily drawn to scale. The FIGS. are only intended to facilitate the description of specific embodiments, and are not intended as an exhaustive description or as a limitation on the scope of the disclosure. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “first” or “second” etc. may be used to distinguish one element from another. The use of the term “first” or “second” should not be construed as in any particular order unless the context clearly dictates otherwise. Further, the singular form of “first” and “second” include plural references unless the context clearly dictates otherwise.

Disclosed herein is a novel blood monitoring system that allows noninvasive, accurate measurement of blood constituents such as blood glucose through a human fingertip. The blood monitoring system includes a finger holder capable of holding or stabilizing human fingers of various sizes. The blood monitoring system may also include an optical bench that can significantly reduce or eliminate optical crosstalk and thus dramatically improve measurement accuracy and efficiency. The blood monitoring system can be self-contained, portable, and is user-friendly. Innovative software is provided to process or calculate measured data. The blood monitoring system enables automatic transmission of stored data to a data management system in a Wi-Fi, Bluetooth or other wireless or wired communication settings.

The disclosed blood monitoring system employs the principle of pulsatile heartbeat spectroscope to extract information from the blood. Pulsatile heartbeat spectroscope is known in the art. Briefly and in general, when light is transmitted through a biological sample such as a human finger, light is absorbed and scattered, or attenuated, by various components of the finger including skin, muscle, bone, fat, interstitial fluid and blood. Light attenuation by a human finger exhibits a cyclic pattern that corresponds to the heartbeat. By way of example, the magnitude of measured photocurrent indicative of the intensity of light transmitted through the human finger exhibits a plot form of pulse waves due to the heartbeat of the user. The plot form includes a plurality of maximums or peaks and a plurality of minimums or valleys. The peak readings of the plot form correspond to when there is a minimal amount of blood in the capillaries of the finger, and the valley readings correspond to when there is a maximal amount of blood in the capillaries of the finger. By using the optical information provided by the peaks and valleys of the cyclic plot, the optical attenuation by major finger constituents that are not in the capillaries such as skin, fat, bones, muscle and interstitial fluids can be excluded, and light that is attenuated by the blood can be measured. Light attenuation caused by blood can be used to determine a glucose level in the blood. For example, by calculating the blood absorption value and comparing the calculated value to predetermined values corresponding to different glucose levels, a blood glucose level of the user can be determined.

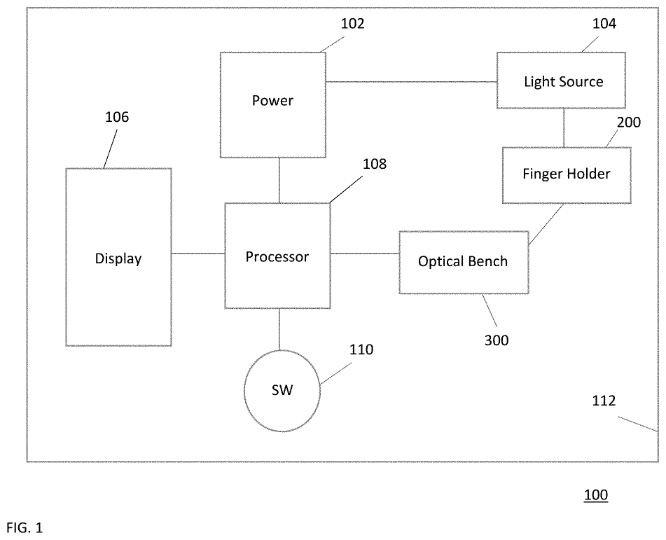

FIG. 1 is a simplified block diagram illustrating some components of an exemplary blood glucose monitoring system 100 according to embodiments of the disclosure. As shown, the blood glucose monitoring system 100 may include a power source 102, a light source 104, a finger holder 200, an optical bench 300, a display 106, a processor 108, and a software program 110, operably and communicably interconnected as shown. The power source 102 provides power to the light source 104 which produces light beams to irradiate a finger held or stabilized in a finger holder 200. The optical bench 300 collects, filters, and detects light attenuated by the finger and generates output signals indicative of the intensity of light detected. The processor 108 executes the software program 110 to calculate a change of light attenuation by the blood in the finger. The change of light attenuation can be used to determine the glucose level in the blood. For example, by comparing the calculated light absorption value to predetermined values corresponding to different glucose levels stored in a memory, a glucose level of the user can be determined. The display 106 can present measurement parameters and/or calculation results. A casing 112 may enclose the power source 102, the light source 104, the finger holder 200, the optical bench 300, the display 106, the processor 108, and other electrical, mechanical, and optical components inside, rendering the blood glucose monitoring system 100 portable or self-contained.

The power source 102 may include a battery such as a thin profile lithium-ion battery. In some embodiments of the disclosure, the power source 102 may be a rechargeable power source. Suitable power source 102 includes but is not limited to MIKROE-1120 and MLP674361 batteries manufactured by Mikro Elektronika of Belgrade, Serbia.

The light source 104 may be an incandescent light source emitting an energy spectrum of a wide range of wavelengths e.g. from 700 to 1600 nanometers. Alternatively, the light source 104 may be a light-emitting diode (LED) emitting an energy spectrum of a narrower range of wavelengths. In some embodiments, the light source 104 may include a plurality of LEDs each emitting an energy spectrum of a specified range of wavelengths. By way of nonlimiting example, an incandescent lamp rated at 5.0 volts and 60 mA emitting a spectrum of wavelengths between 700 to 1600 nanometers can be used. Suitable light source 104 includes but is not limited to Model No. 7683 incandescent lamp manufactured by JKL Components Corporation of Pacoima, Calif., which has an average life expectancy of 1000,000 hours.

The processor 108 can be any suitable microprocessor which incorporates the functions of a computer's central processing unit (CPU) on an integrated circuit. The processor 108 may be a single CPU or dual CPU or dual core microprocessor. By way of example, the processor 108 can be Model No. STM32F407/417 microprocessor manufactured by ST Microelectronics Inc. of Santa Clara, Calif. The STM32F407/417 microprocessor provides high level of integration and performance and includes embedded memories and rich peripheral set inside packages as small as 10×10 mm. The STM32F407/417 microprocessor provides the performance of the Cortex™-M4 core (with floating point unit) running at 168 MHz. It will be appreciated by one of ordinary skill in the art that other suitable microprocessors can also be used.

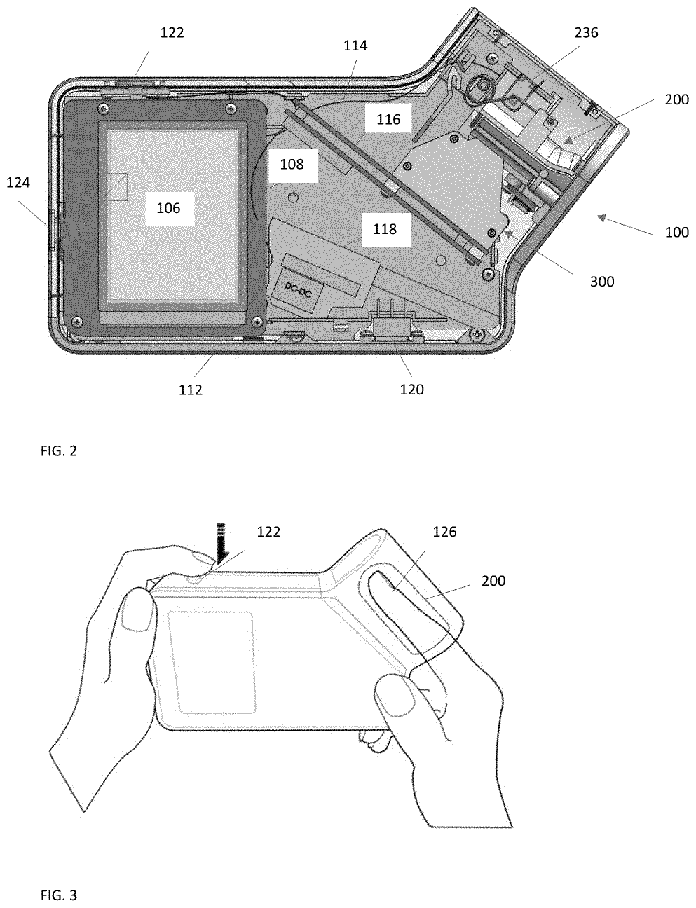

FIG. 2 is a cutaway view of an exemplary glucose monitoring system 100 showing arrangements of some components according to embodiments of the disclosure. The finger holder is indicated at 200. The light source 104 (shown in FIG. 1 but not in FIG. 2) is enclosed in a light source housing 236 on the finger holder 200. The optical bench is indicated at 300. The display is indicated at 106. The processor or CPU board including a memory, a processor, and other electrical circuitries is indicated at 108. While not visible or shown in FIG. 2, the power source can be e.g. disposed below the CPU board 108, supplying power to the light source 104 via a flexible cable 114 and to other components via buses or flexible cables. Reference 116 indicates an operational amplifier board coupled to the optical bench 300. Reference 118 indicates an analog-to-digital converter board. A power switch is indicated at 120, a glucose measurement button is indicated at 122, and a data transmission port such as a USB port is indicated at 124. The casing 112 encloses the components of the monitoring system inside. FIG. 3 schematically shows that a user is taking blood glucose measurement by inserting a finger 126 in the finger holder 200 and pressing the measurement button 122.

Finger Holder

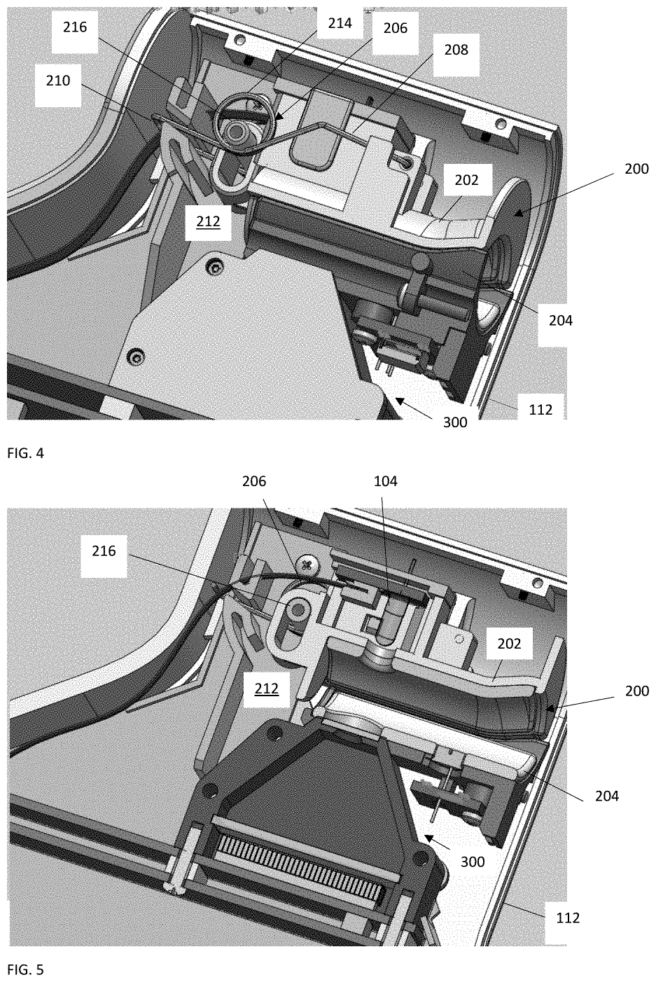

Referring now to FIGS. 4-14, embodiments of a finger holder will now be described. FIGS. 4 and 5 schematically shows an exemplary finger holder 200 included in a blood glucose monitoring system according to embodiments of the disclosure. As shown, the exemplary finger holder 200 in general includes an upper (first) holder portion 202, a lower (second) holder portion 204, and a torsion spring 206 coupling the upper and lower holder portions 202, 204. The torsion spring 206 may include a first arm 208 urging the upper holder portion 202 to the lower holder portion 204, a second arm 210 anchored to a support member 212, and a spring coil 214. The spring coil 214 of the torsion spring 206 may be retained by a retaining post 216 secured to the support member 212, allowing the upper holder portion 202 to rotate about the retaining post 216. In embodiments of the disclosure, the finger holder 200 may be enclosed inside the casing 112. The lower holder portion 204 may be fixedly attached to the casing 112, whereas the upper holder portion 202 may be movable relative to the lower holder portion 204 to accommodate fingers of various sizes.

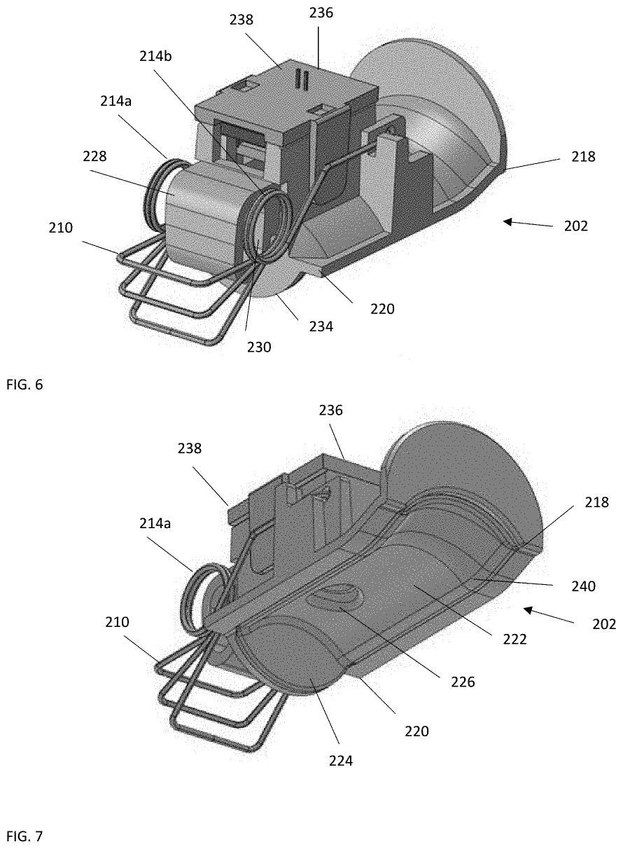

FIG. 6 is a top perspective view, FIG. 7 a bottom perspective view of an exemplary upper (first) holder portion 202 according to embodiments of the disclosure. As shown, the upper holder portion 202 include a first end 218, a second end 220, and an inner surface 222 extending from the first end 218 to the second end 220. The contour of first end 218 of the upper holder portion 202 and the first end 242 of the lower holder portion 204, to be described below, forms an opening to admit a finger. The inner surface 222 of the upper holder portion 202 may be contoured to conform to a portion of the finger. At the second end 220 of the upper holder portion 202, a stopper 224 may be provided to prevent the fingertip from extending beyond the finger holder 200. An aperture 226 is provided in the upper holder portion 202 to allow light emitted from the light source passing through to irradiate the finger.

The upper holder portion 202 may include a spring-retaining or retaining structure 228 at the second end 220. The retaining structure 228 may be provided with a through slot or opening 230 receiving the retaining post 216 (FIGS. 4 and 5). The through slot 230 may have an elongate profile, allowing the upper holder portion 202 to translate or move up and down relative to the lower holder portion 204, thereby increasing or decreasing the space between the inner surfaces of the upper and lower holder portions 202, 204 to accommodate fingers of different sizes. The spring coil 214 of the torsion spring 206 may include a first coil section 214a and a second coil section 214b spaced apart and connected by the second arm 210, which may be anchored to an anchor support. The first and second spring coil sections 214a, 214b may be disposed at either side of the retaining structure 228 and retained by the retaining post 216 (FIGS. 4 and 5).

Still referring to FIGS. 6 and 7, the upper holder portion 202 may also include an enclosure 236 enclosing a light source 104 (FIG. 5). The enclosure 236 may include an aperture that aligns with the aperture 226 in the upper holder portion 202 to provide an optical path for light beams. The enclosure 236 may include a cover 238 onto which the light source 104 and an electrical circuitry board for the light source may be mounted.

Referring to FIG. 7, the upper holder portion 202 may include a finger pad 240. The finger pad 240 may be made of a material that can properly stabilize or encapsulate the finger or provide comfort to the patient. The finger pad 240 may also block or minimize stray light that would otherwise affect measurement. Suitable materials for the finger pad 240 include silicone, rubber, foam, or the like. By way of nonlimiting example, thermoplastic materials such as polyurethane (Gemothane®), liquid silicone rubber may be used for constructing the finger pad 240. The finger pad 240 may be attached to the inner surface of the upper holder portion 202 by e.g. adhesives or other suitable means. Alternatively, the finger pad 240 may constitute a part of the inner surface 222 of the upper holder portion 202.

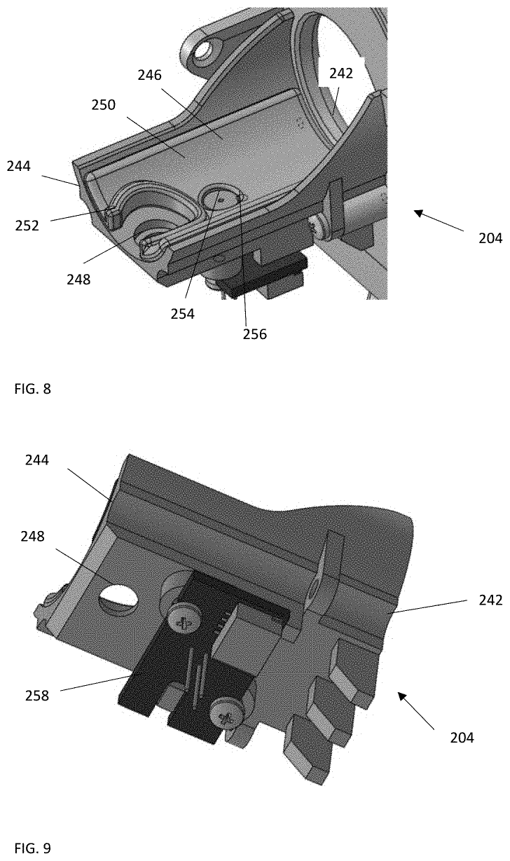

FIG. 8 is a top perspective view, FIG. 9 a bottom perspective view of an exemplary lower (second) holder portion 204 according to embodiments of the disclosure. As shown, the lower holder portion 204 includes a first end 242, a second end 244, and an inner surface 246 extending from the first end 242 to the second end 244. The first end 242 of the lower holder portion 204 and the first end 218 of the upper holder portion 202, described above, forms an opening for admitting a finger. The inner surface 246 of the lower holder portion 204 may be contoured to conform to a portion of the finger. An aperture 248 is provided in the lower holder portion 204 to allow light attenuated by or transmitted through the finger to exit to an optical bench, to be described below.

The lower holder portion 204 may include a finger pad 250. The finger pad 250 may be made of a material that can properly stabilize or encapsulate the finger or increase patient's comfort. The finger pad 250 may also block or minimize stray light that would affect measurement. Suitable materials for the finger pad 250 include silicone, rubber, foam, or the like. By way of nonlimiting example, thermoplastic materials such as polyurethane (Gemothane®), liquid silicone rubber may be used for constructing the finger pad. The finger pad 250 may be attached to the inner surface of the lower holder portion 204 by e.g. adhesives or other suitable means. Alternatively, the finger pad 250 may constitute a part of the inner surface of the lower holder portion 204. In some embodiments, a ridge 252 along the aperture 248 of the lower holder portion 204 may be provided to help position or stabilize a fingertip at the aperture 248. Alternatively, in some embodiments, an indentation generally conforming to the fingertip may be provided near the aperture to help position or stabilize the fingertip.

Still referring to FIGS. 8 and 9, in some embodiments, the lower holder portion 204 may include a temperature sensor 254 for detecting the temperature of the patient's finger. The temperature sensor 254 may be disposed in an aperture 256 in the lower holder portion 204. An electrical circuitry board 258 for the temperature sensor 254 may be attached to the bottom of the lower holder portion 204 by e.g. screws, pins or the like. The temperature sensor 254 may be integrated circuit sensors or electrical temperature sensors. By way of nonlimiting example, the temperature sensor 254 may be an infrared temperature sensor.

The lower holder portion 204 may be fixedly attached to a support body such as the casing 112 (FIGS. 4 and 5) with screws or other mounting structures. In use, the patient's finger can be held by the lower holder portion 204. The upper holder portion 204 can pivot about the retaining post 216 to increase or decrease the opening formed by the first ends 218, 242 of the upper and lower holder portions 202, 204, or move up or down to increase or decrease the space between the upper and lower holder portions 202, 204. Advantageously, an adjustable finger holder 200 disclosed herein can accommodate patient's fingers of different sizes, which can be received and gripped by the upper and lower holder portions 202, 204 with comfort. The elongate inner surfaces 222, 246 of the first and second holder portions 202, 204 can uniformly distribution the urging pressure applied by the torsion spring 206, improving measurement accuracy. Fingers of various sizes can be conformably stabilized in the finger holder 200 but not squeezed or clamped too tightly. Clamping the finger too tightly would cause patient discomfort and measurement inaccuracy.

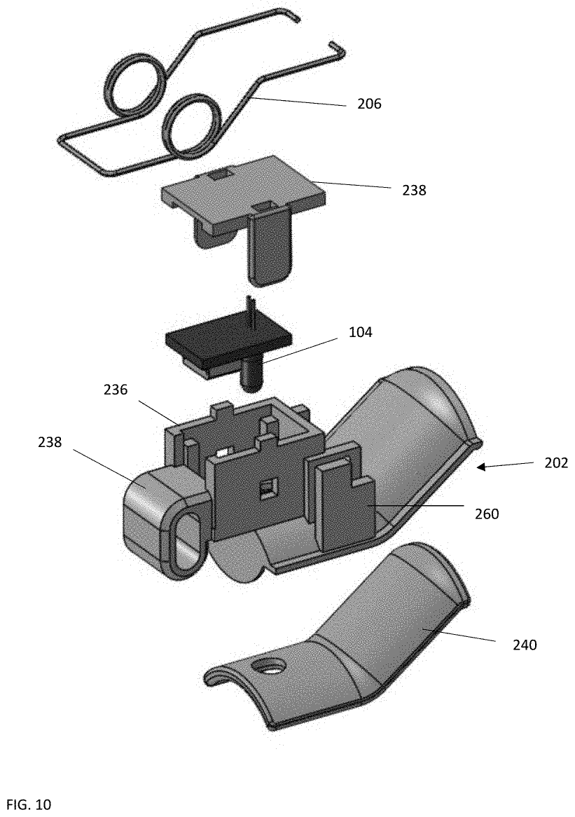

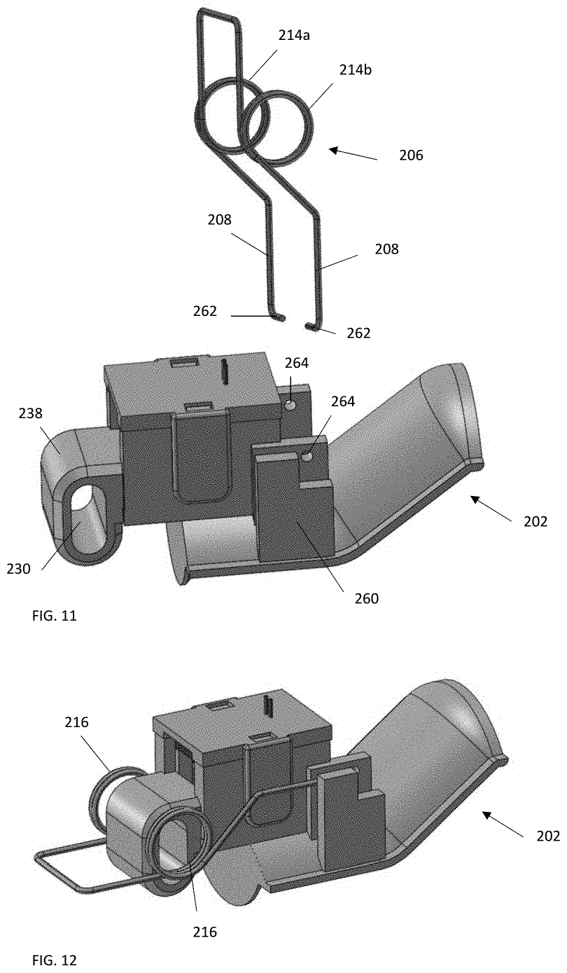

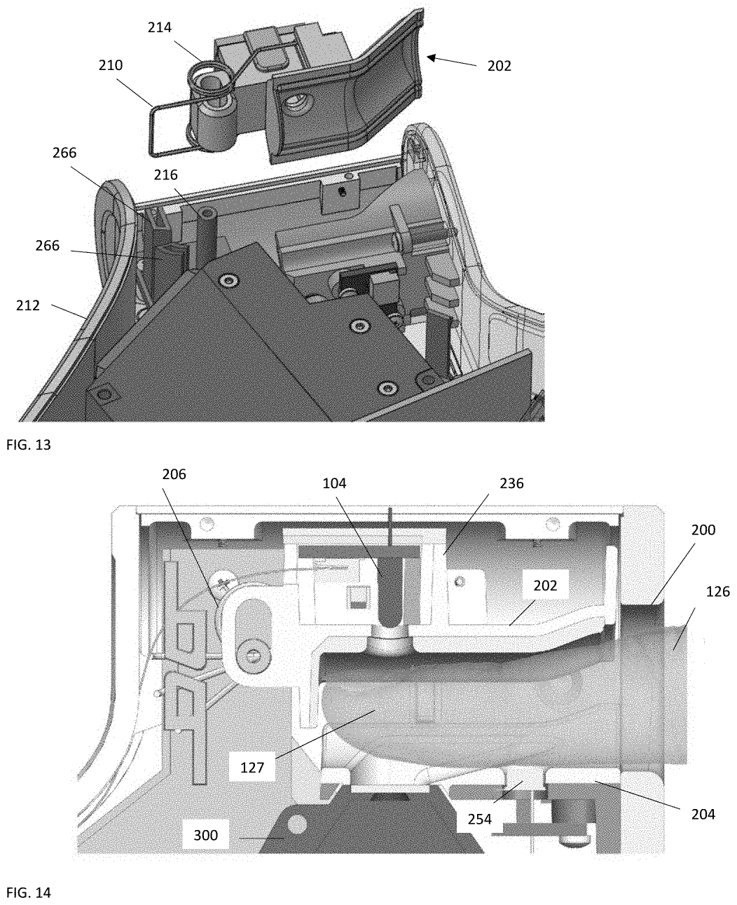

FIG. 10 is an exploded view showing individual parts of a torsion spring 206, a cover 238 for a light source enclosure 236, a light source 104, an upper holder portion 202 including a spring retaining structure 238, a light source enclosure 236, spring retaining walls 260, and a finger pad 240. FIG. 11 and FIG. 12 show assembling of the torsion spring 206 to the upper holder portion 202. FIG. 13 shows assembling of the upper holder portion 202-torsion spring 206 in a blood glucose monitoring system.

As shown in FIGS. 11 and 12, the first arm 208 of the torsion spring 206 may include two hooks or ends 262, which can be inserted into the holes 264 in the retaining walls 260 on either side of the upper holder portion 202. The torsion spring 206 can be then swung down to align the spring coils 214a, 214b with the through slot 230 of the spring retaining structure 238. The retaining walls 260 may constrain the first arm 208, keeping the ends 262 of the first arm 208 in the holes 264 when the upper holder portion 202 pivots or moves. The upper holder portion 202 with the torsion spring 206 can be then assembled in the monitoring system as shown in FIG. 13, with the spring coils 214 being retained by the retaining post 216, which is fixed on the support member 212, and the second arm 210 being anchored to the anchors 266, which are fixed on the support member 212. The support member 212 may be part of the casing 112, or a separate support frame attached to the casing 112.

FIG. 14 schematically shows a finger 126 inserted into the finger holder 200, with the fingertip 127 being aligned with the apertures in the light source enclosure 236 and in the upper and lower holder portions 202, 204. The torsion spring 206 applies a force on the upper holder portion 202, causing the finger to be gripped or stabilized in the finger holder 200 during measurement. Light beams from the light source 104 passes through the aperture in the upper holder portion 202, irradiating the fingertip 127 Light attenuated by the fingertip exits the aperture in the lower holder portion 204 and detected by a detector array in the optical bench 300, to be described in greater detail below. The temperature sensor 254 measures the patient's temperature through the patient finger 126.

Optical Bench

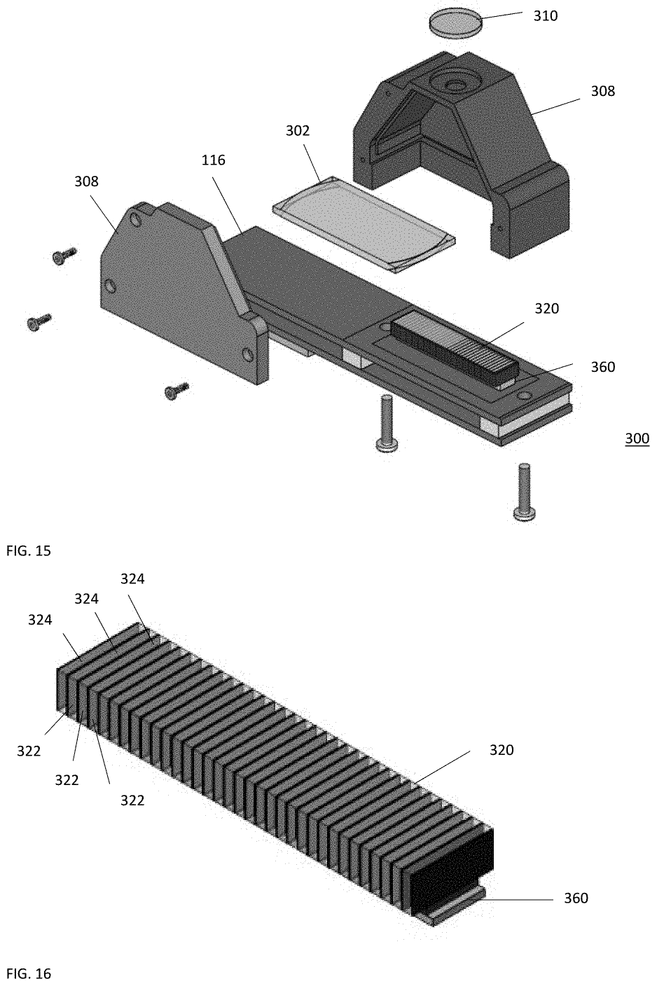

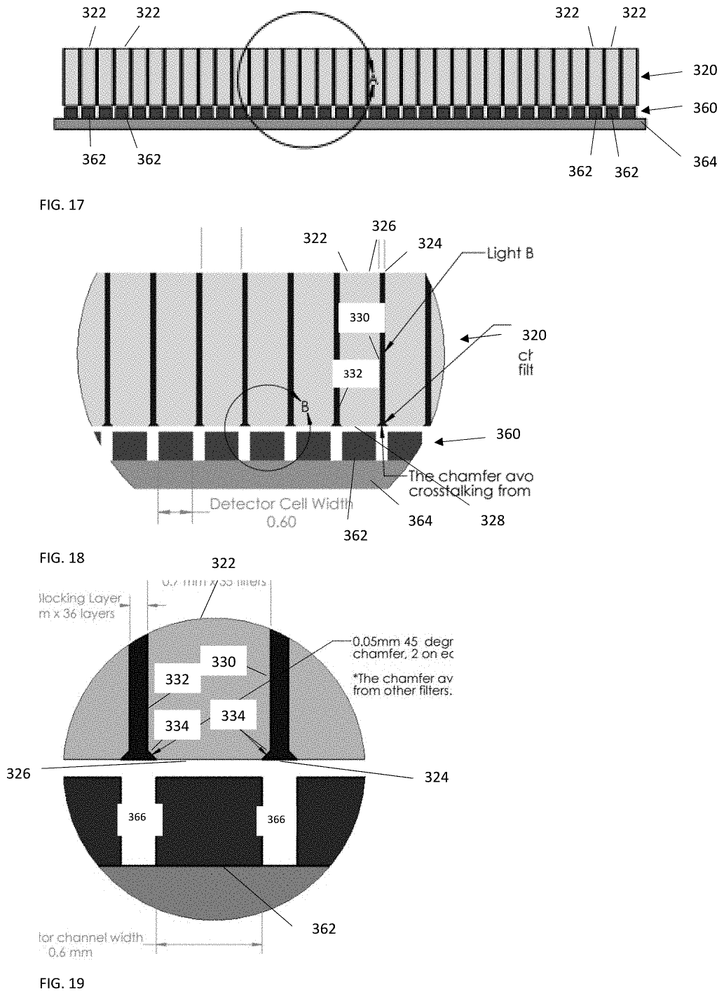

Referring to FIGS. 15-21, embodiments of an optical bench or apparatus 300 will now be described. FIG. 15 is an exploded view of an exemplary optical bench 300 according to embodiments of the disclosure. As shown, the optical bench 300 may include a collimation lens 302, a bandpass filter array 320, a detector array 360, and a housing 308 enclosing the collimation lens 302, the bandpass filter array 320, the detector array 360, and optionally a portion of an operational amplifier board 116. The housing 308 may include an optical window 310 allowing light to pass through. An assembled optical bench 300 is shown in FIGS. 2 and 4. A cutaway view of an optical bench 300 is shown in FIG. 5.

Referring to FIGS. 15-21, the collimation lens 302 collimates light attenuated e.g. by a finger, which may diffuse in various directions. Any suitable collimation lens may be used. In an exemplary embodiment, a Fresnel lens may be used as the collimation lens 302. A Fresnel lens, which may be made thin or compact, includes a series of concentric grooves replacing the curved surface of a conventional optical lens. Alternatively, a singlet, doublet or aspheric lens can be used as collimation lens 302.

The bandpass filter array 320 selectively transmits collimated light from the collimation lens 302. As shown in greater detail in FIGS. 16-19, the bandpass filter array 320 may include a plurality of bandpass filters 322 arranged side by side in an array. Between neighboring bandpass filters 322, light blocking layers 324 are disposed to suppress light crosstalk among neighboring bandpass filters 322. The light blocking layers 324 may be made of light absorbing materials such as polycarbonate or other suitable polymer materials. Alternatively, the light blocking layers 324 may be made of light reflective materials such as Mylar or the like. The light blocking layers 324 may prevent light in a bandpass filter from escaping to neighboring bandpass filters, leading light transmitted by a particular bandpass filter 322 to a corresponding light detection element 362 directly underneath the bandpass filter.

The detector array 360 detects light transmitted through the bandpass filter array 320. The detector array 360 may include a plurality of detection elements 362. Each of the detection elements 362 may include a photosensitive element configured to convert light into electrical signals and a switching element for access to the electrical charges by readout electronics. The photosensitive element may be a photodiode, a photoconductor, a photogate, or a phototransistor etc. The switching element may be a thin film transistor (TFT) or other switching elements such as organic transistors, charge coupled devices (CCDs), CMOS, metal oxide transistors, or transistors made of other semiconductor materials, and/or switching diodes. The TFTs may be amorphous silicon (a-Si), metal oxide or polycrystalline silicon TFTs. The photosensitive elements and switching elements may be formed on a substrate 364 by any methods known in the art, and thus their detail description is omitted here in order to focus on description of embodiments of this disclosure. The detector array 360 may be optically coupled to the bandpass filter array 320 by optically transparent adhesive or other suitable means.

The plurality detection elements 362 of the detector array 360 and the plurality of filters 322 of the bandpass filter array 320 may be arranged such that each detection element 362 corresponds to one of the plurality of bandpass filters 322. Each of the detection elements 362 detects light transmitted by one of the bandpass filters 322 and generates current signals that is proportional to the power of light received by the detection element 362. The current signal generated by the detection element 362 may be converted to another form of signal such as an analog voltage signal or a digital signal by an operational analog-digital converter.

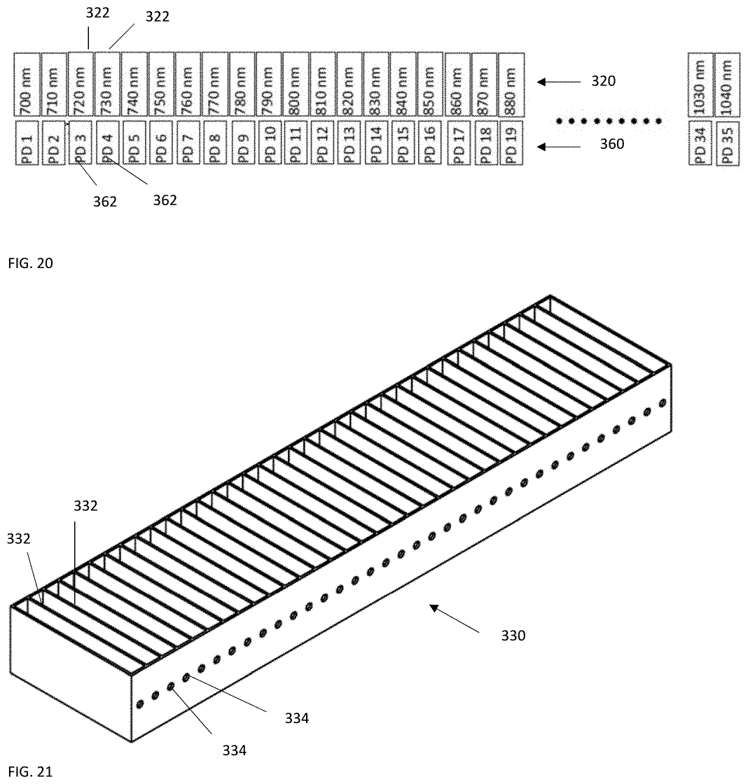

Referring to FIG. 20, each of the plurality of bandpass filters 322 may be configured to transmit a selected band of wavelengths. In the following description and appended claims, the term “transmission center wavelength” may be used to refer to a center wavelength of an optical spectrum that a bandpass filter transmits. As shown in FIG. 20, each of the plurality of bandpass filters 322 may have a unique transmission center wavelength. For example, a bandpass filter 322 may have a transmission center wavelength different from a neighboring bandpass filter.

The bandpass filters 322 may be arranged such that the transmission center wavelengths of the plurality of bandpass filters are spread across a wavelength range. By way of example, the transmission center wavelengths of the plurality of bandpass filters may be spread across a wavelength range from 700 to 1040 nanometer, as shown in FIG. 20. The transmission center wavelengths of the plurality of bandpass filters 322 may be spread across a wavelength range in a successively increased or decreased order. In some embodiments, the transmission center wavelengths of the plurality of bandpass filters may be spread across wavelength range by a constant wavelength step such as 10, 20, 30, 40, or 50 nanometers and so on, or by an integer multiple of a wavelength step.

In the nonlimiting exemplary embodiment shown in FIG. 20, the bandpass filter array 320 includes 35 bandpass filters 322 each having a unique transmission center wavelength. The transmission center wavelengths of the 35 bandpass filters are spread from 700 to 1040 nanometers with a wavelength step of up to 10 nanometers. It should be noted that FIG. 20 is provided for illustration purpose and the bandpass filter array 320 according to embodiments of the disclosure may include more or less than 35 filters. Further, the bandpass filters 322 may be arranged in any desirable orders and thus their transmission center wavelengths are not necessarily in a successively increased or decreased order.

Still referring to FIG. 20, each of the bandpass filters 322 corresponds to one of the light detection elements 362 located below each bandpass filter 322. By way of example, light detection element PD1 receives light transmitted from the bandpass filter having a transmission center wavelength of 700 nm. Light detection element PD4 receives light from the bandpass filter having a transmission center wavelength of 730 nm. The design and construction of the disclosed bandpass filter array 320 can prevent or suppress light transmitted from e.g. the bandpass filter having a transmission center wavelength of 780 nm from propagating to the adjacent light detection elements PD8 or PD10, thereby preventing or reducing potential optical crosstalk.

Referring to FIGS. 18 and 19, each of the plurality of bandpass filters 322 may include a first end 326 facing incident light, a second end 328 exiting light transmitted through the filter, a first side 330 and a second side 332 extending from the first end 326 to the second end 323. According to embodiments of the disclosure, the first and second sides 330, 332 of at least some of the plurality of bandpass filters 322 are chamfered at the second end 328 of the at least some of the plurality of bandpass filters 322. In some embodiments, the first and second sides 330, 332 of each of the plurality of bandpass filters 322 are chamfered at the second end 328 of each of the plurality of bandpass filters 322. Advantageously, the chamfered configuration at the bottom of a bandpass filter, along with the light blocking layers, can prevent light from the bandpass filter from traveling across neighboring light detection elements, thereby further suppressing or reducing any potential optical crosstalk.

In the following description and appended claims, the term “chamfer angle” may be used to refer to an angle between the first or second side surface 330, 332 of a bandpass filter 322 and the beveled surface 334 at the bottom or second end 328 of the filter 322. According to embodiments of the disclosure, the chamfer angle of a bandpass filter 322 may be any angle in the range from 10 to 80 degrees. In some embodiments, the chamfer angle may be any angle in the range from 30 to 60 degrees. In an exemplary example, the chamfer angle may be about 45 degrees. In another exemplary example, the chamfer angle of a bandpass filter may be about 60 degrees. As illustrated in FIG. 19, the light blocking layer 324 on the chamfered portion of a bandpass filter 322 will block light in a direction that would propagate into neighboring light detection elements.

In embodiments of the disclosure, the light blocking layer 324 at the bandpass filters 322 may extend at least partially or all the way down to the space 366 between light detection elements 362. In alternative embodiments, the detector array 360 may include a plurality of light blockers (not shown) each may be disposed in the space 366 between two neighboring detection elements 362. The light blockers may be made of light absorbing materials that absorb light and thus help prevent light from entering into neighboring detection elements.

FIG. 21 is a perspective view of an exemplary filter holder 330 for holding bandpass filters 322 according to embodiments of the disclosure. As shown, the holder 330 may include a plurality of walls 332 defining an array of cavities for holding a plurality of bandpass filters 322 disposed therein. A plurality of apertures 334 may be provided in the side walls for receiving e.g. adhesives to secure the bandpass filters 322 to the holder 330. In some embodiments, the holder 330 may be constructed from plastic, metal, or other suitable light blocking materials. An exemplary light blocking material includes polycarbonates. In some embodiments, the side walls 332 of the holder may be made from a light blocking material to function as light blocking layers of the bandpass filter array 320.

Embodiments of a glucose monitoring system have been described. Those skilled in the art will appreciate that various other modifications may be made within the spirit and scope of the invention. All these or other variations and modifications are contemplated by the inventors and within the scope of the invention.

1. A finger holder, comprising:
a first holder portion having a first end, a second end, and an inner surface extending from the first end to the second end;
a second holder portion having a first end, a second end, and an inner surface extending from the first end to the second end, the first ends of the first and second holder portions forming an opening to admit a finger to be received between the inner surfaces of the first and second holder portions; and
a torsion spring comprising a first arm coupled to the first holder to urge the first holder portion to the second holder portion, a second arm anchored to a support member, and a spring coil retained by a retaining post on the support member allowing the first holder portion to rotate about the retaining post thereby increasing or decreasing a size of the opening admitting the finger.

2. The finger holder of claim 1, wherein the first holder portion further comprises a retaining structure at the second end provided with a through slot for receiving the retaining post, the through slot of the retaining structure having a size allowing the first holder portion to translatively move relative to the second holder portion and the retaining post, thereby increasing or decreasing a space between the inner surfaces of the first and second holder portions.

3. The finger holder of claim 2, wherein the spring coil comprises a first coil section and a second coil section spaced apart and connected by the second arm, wherein the first and second coil sections are disposed outside of the through slot of the retaining structure and retained by the retaining post.

4. The finger holder of claim 1, wherein the first holder portion further comprises an enclosure enclosing a light source.

5. The finger holder of claim 1, wherein the first holder further comprises a stopper at the second end preventing the finger from extending beyond the finger holder.

6. The finger holder of claim 1, wherein the second holder portion further comprises a temperature sensor detecting a temperature of the finger.

7. The finger holder of claim 1, wherein the first holder portion is provided with an aperture allowing light passing through to irradiate the finger, and the second holder portion is provided with an aperture allowing light attenuated by the finger to exit through.

8. The finger holder of claim 7, wherein the second holder portion further comprises a ridge on the inner surface along the aperture of the second holder portion to position or stabilize a fingertip at the aperture of the second holder portion.

9. The finger holder of claim 1, wherein the first and second holder portions further comprise a finger pad respectively, the finger pad is constructed from a material comprising polyurethane or liquid silicone rubber.

10. A blood monitoring system, comprising:
a light source producing light beams;
a finger holder configured to hold a finger to be irradiated by the light beams;
a detector array detecting light attenuated by the finger and generating output signals indicative of intensity of the light detected; and
a processor determining a characteristic of a blood constituent in the finger based on the generated output signals,
wherein the finger holder comprises:
a first holder portion having a first end, a second end, and an inner surface extending from the first end to the second end;
a second holder portion having a first end, a second end, and an inner surface extending from the first end to the second end, the first ends of the first and second holder portions forming an opening to admit the finger to be received between the inner surfaces of the first and second holder portions; and
a torsion spring comprising a first arm coupled to the first holder to urge the first holder portion to the second holder portion, a second arm anchored to a support member, and a spring coil retained by a retaining post on the support member allowing the first holder portion to rotate about the retaining post thereby increasing or decreasing a size of the opening admitting the finger.

11. The blood monitoring system of claim 10, further comprising a casing enclosing the light source, the finger holder, the detector array, and the processor.

12. The blood monitoring system of claim 10, wherein the light source is an incandescent light source.

13. The blood monitoring system of claim 10, wherein the processor comprises a duo core processor.

14. The blood monitoring system of claim 10, wherein the first holder portion further comprises a retaining structure at the second end provided with a through slot for receiving the retaining post, the through slot of the retaining structure having a size allowing the first holder portion to translatively move relative to the second holder portion and the retaining post, thereby increasing or decreasing a space between the inner surfaces of the first and second holder portions.

15. The blood monitoring system of claim 14, wherein the spring coil comprises a first coil section and a second coil section spaced apart and connected by the second arm, wherein the first and second coil sections are disposed outside of the through slot of the retaining structure and retained by the retaining post.

16. The blood monitoring system of claim 10, wherein the first holder portion further comprises an enclosure enclosing the light source.

17. The blood monitoring system of claim 10, wherein the second holder portion further comprises a temperature sensor detecting a temperature of the finger.

18. The blood monitoring system of claim 10, wherein the first holder portion is provided with an aperture to allow the light beams passing through to irradiate the finger, and the second holder portion is provided with an aperture to allow light attenuated by the finger to exit through.

19. The blood monitoring system of claim 18, wherein the second holder portion further comprises a ridge on the inner surface along the aperture of the second holder portion to position or stabilize a fingertip at the aperture of the second holder portion.

20. The blood monitoring system of claim 10, wherein the first and second holder portions further comprise a finger pad respectively, the finger pad is constructed from a material comprising polyurethane or liquid silicone rubber.

21-51. (canceled)
